(19) United States
(12) Patent Application Publication
(10) Pub. No.: US 2006/0063835 A1
(43) Pub. Date: Mar. 23, 2006

(54) Composition based on triethyl citrate for the treatment of bacterial infections of the skin

(76) Inventor: Gianfranco De Paoli Ambrosi, Salo (Brescia) (IT)

Correspondence Address: MCGLEW & TUTTLE, PC, P.O. BOX 9227, SCARBOROUGH STATION, SCARBOROUGH, NY, 10510-9227, US

(21) Appl. No.: 10/526,152

(22) PCT Filed: Sep. 2, 2003
(86) PCT No.: PCT/IT03/00530

§ 371 Date: Feb. 24, 2005

(30) Foreign Application Priority Data
Sep. 2, 2002 (IT) BS2002A000078

(51) Int. Cl. A61K 31/685; A61K 31/525; A61K 31/496; A61K 31/366; A61K 31/20; A61K 31/225

(52) U.S. Cl. 514/547; 514/559; 514/251; 514/350; 514/355; 514/560; 514/78; 514/546; 514/460; 514/254.07

(57) ABSTRACT

This invention concerns a composition for topical use containing as an active ingredient triethyl citrate either pure or in combination with synergists, and the pharmaceutical or cosmetic use of the composition, on its own or in association with an antibiotic, at least in the treatment of cutaneous pathologies directly or indirectly affected by bacterial infections.

### FIELD OF THE INVENTION

This invention concerns a new composition for cosmetic or pharmaceutical purposes, for external use, to be applied either on the cutis, whether integral or damaged, or on the mucous membrane, in order to improve all cutaneous pathologies both directly and indirectly affected by bacterial infections, such as for example superficial primary pyodermia and impetigo vulgaris and other common dermatitis infections, such as for example atopic dermatitis and the various forms of eczema.

### STATE OF THE ART

Antibiotic therapy for topical use is used preferably in the dermatological field in that it allows the use of sufficient quantities of active principle in the area directly affected by the infectious process, avoiding the risks connected with systematic antibiotic therapy.

Triethyl citrate, that is a triethyl ester of citric acid, is well known and used in the cosmetic sector for the treatment of ageing of the skin (U.S. Pat. No. 5,686,489 dated 21 Nov. 1997), but it has never been either proposed or suggested as an active ingredient for the treatment of bacterial cutaneous infections, neither alone or in synergy with other substances.

Now, following specific research and experiments carried out by the inventor, it has become clear that the active ingredient, triethyl citrate taken into consideration herein, carries out an activity, comparable to and which can be placed over substances possessing antibiotic, antiseptic and disinfectant activities, without generating bacterial resistance phenomena (on the contrary to the more common antibiotics).

### OBJECTS AND SUMMARY OF THE INVENTION

This invention is based on the results of this research, and therefore its primary object is to propose the use of a new active principle useful at least in the cure of cutaneous pathologies involving infections having bacterial origins.

A further object of the invention is to provide an active principle for the formulation of products, both cosmetic and pharmaceutical, to be used locally in the treatment of cutaneous infections caused by bacteria, without producing bacterial resistance.

Yet another scope of the invention is to provide an active composition for the cure of cutaneous infections and which, advantageously, used in combination with antibiotics, antiseptics and disinfectants is able to prevent the setting in of bacterial resistance phenomena.

These aims are achieved, according to the invention, with a composition for cosmetic and pharmaceutical use containing triethyl citrate, as an active ingredient, pure or in association with synergists.

### DETAILED DESCRIPTION OF THE INVENTION

In this invention and for the use given above, triethyl citrate may be used pure with suitable supports or vehicles, or better formulated with other chemical substances, such as synergists, additives and excipients as a percentage by weight from 0.1 to 99.9%, preferably from 0.5 to 50%, and better still from 5.0 to 15% on the basis of the final formulation, for both cosmetic and pharmaceutical preparations for local use.

Accordingly, the active ingredient represented by triethyl citrate can be used, for example, in combination with substances which are part of the chemical group which include carboxylic acids, hydroxyacids, vitamins, amino acids, bioflavonoids, oligoelements, essential fatty acids and relative esters, antibiotics, sulphamides, disinfectants. Oleic, linolic and linolenic acid ethyl esters and other compounds such as for example erythromycin, clindamycin, metronidazole, gentamicin, fusidic acid, econazole, ketoconazole, mupirocin, hydrogen peroxide, benzoyl peroxide, cetylpyridinium, silver and relative salts, both organic and inorganic.

Synergists are understood to be for example: trans-retinal acid, retinol, retinaldehyde, tocopherol, ascorbic acid, p-aminobenzoic acid, rutin, β-Carotene, tiamin, riboflavin, pyridoxine, pyridoxale, niacin, nicotinic acid, nicotinamide, pantothenic acid, pantenol, glucosamine, aceylglucosamine, folic acid, lecithin, phosphplipids such as, for example phosphatidylcholine, phosphatidylethanolamine, phosphatidic acid, lyso-phosphatidylcholine, hydroquinone, oleic acid, linoleic acid, linolenic acid, ethyl oleate, ethyl linolenate, ethyl linoleate, Kojic acid, ascorbyl glucoside, erythromycin, clindamycin, metronidazole, gentamicin, fusidic acid, econazole, ketoconazole, mupirocin, neomocin, stretomicin, hydrogen peroxide, benzoil peroxide, cetylpyridinium, benzalkonium, chlorhexidin and relative salts and esters, silver and relative salts, both organic and inorganic, hydroxyacids and β hydroxyacids, both mono and bi carboxyls, such as glycolic acid, lactic acid (in the dextro and levorotatory forms and in racemic mixtures) hydroxybutyric acid (in the dextro and levorotatory forms and in racemic mixtures), mandelic acid (in the dextro and levorotatory forms and in racemic mixtures), tartaric acid (in the dextro and levorotatory forms and in racemic mixtures), malic acid (in the dextro and levorotatory forms and in racemic mixtures), salicylic acid, 3-hydroxybenzoin acid, 4-hydroxybenzoin acid, cysteine, acetyl cysteine, glycine, used singularly or in association with one or more including the relative salts, esters and amides and the relative D-L-DL forms.

The components of this group of substances can be used in association with triethyl citrate in a percentage by weight from 0.01% to 50% in weight, preferably from 0.5 to 15%.

The following EXAMPLES of preparations illustrate even further the efficacy of the composition of this invention which contains triethyl citrate as an active ingredient.

Triethyl citrate, possibly associated with appropriate synergists as described above, can be used in formulations for external use, such as a water emulsion in oil, oil emulsions in water, single phase solutions, dual phase pseudo-solutions, single phase gels, dual phase gels, anhydrous ointments and in powder form etc, using appropriate supports and vehicles. EXAMPLES of preparations based on triethyl citrate base.

### PREPARATION 1

| No. | Description | |
|---|---|---|
| 01 | Triethyl citrate | 100 |

Preparation method: use as it is

### PREPARATION 2

| No. | Description | |
|---|---|---|
| 01 | Triethyl citrate | 20.00 |
| 02 | Erythromycin | 2.00 |
| 03 | Ethyl alcohol | 60.00 |
| 04 | Deionised water | 18.00 |

Preparation method: dissolve 02 in 03; mix 01 in the solution obtained; then add 04

### PREPARATION 3

| No. | Description | |
|---|---|---|
| 01 | Triethyl citrate | 6.00 |
| 02 | Salicylic acid | 0.50 |
| 03 | Ethyl alcohol | 60.00 |
| 04 | Deionised water | 33.50 |

Preparation method: dissolve 02 in 03; mix 01 in the solution obtained; then add 04

### PREPARATION 4

| No. | Description | |
|---|---|---|
| 01 | Triethyl citrate | 25.00 |
| 02 | Retinic acid | 0.025 |
| 03 | Ppg - 15 stearyl ether -as needed | 100 |

Preparation method: dissolve 02 in 03; mix 01 in the solution obtained;

### PREPARATION 5

| No. | Description | |
|---|---|---|
| 01 | Triethyl citrate | 95.00 |
| 02 | Ethyl linoleate | 5.00 |

Preparation method: dissolve 02 in 01;

### PREPARATION 6

| No. | Description | |
|---|---|---|
| | A) | |
| 01 | Triethyl citrate | 10,000 |
| 02 | Steareth-2 | 3,000 |
| 03 | Steareth-21 | 2,000 |
| 04 | Vaseline oil | 1,000 |
| 05 | Stearic acid | 5,000 |
| | B) | |
| 06 | Preservatives | As needed |
| 07 | Glycerol | 4,000 |
| 08 | Deionised water | As needed |
| | | 100 |

Preparation method: the ingredients (A) and ingredients (B) are heated separately at 70° C. Then ingredients (B) are added to ingredients (A) mixing until a well amalgamated mixture in the form of an emulsion for topical use is obtained.

### PREPARATION 7

| No. | Description | |
|---|---|---|
| 01 | Triethyl citrate | 5,000 |
| 02 | Chlorhexidine gluconate | 0,250 |
| 03 | Idrossietil cellulose | 1,000 |
| 04 | Deionised water as needed | 100 |

Preparation method: dissolve 01+02 in 04; in the solution obtained disperse 03 until complete solvation and formation of a gel.

1. A composition for topical use for curing cutaneous pathologies, characterized in that it contains as an active ingredient triethyl citrate either pure or in combination with synergists.

2. A composition according to claim 1, which contains triethyl citrate in a percentage by weight of 0.1 to 99.9. preferably from 5 to 50 percent.

3. A composition according to claim 2, which contains triethyl citrate in a percentage by weight of 5.0 to 50.0 percent.

4. A composition according to claim 1, containing the active ingredient represented by triethyl citrate in association with at least one of the additional substances chosen between trans-retinal acid, retinol, retinaldehyde, tocopherol, ascorbic acid, p-aminobenzoic acid, rutin, β-Carotene, tiamin, riboflavin, pyridoxine, pyridoxale, niacin, nicotinic acid, nicotinamide, pantothenic acid, pantenol, glucosamine, aceylglucosamine, folic acid, lecithin, phosphplipids such as, for example phosphatidylchohne, phosphatidylethanolamine, phosphatidic acid, lyso-phosphatidylcholine, hydroquinone, oleic acid, linoleic acid, linolenic acid, ethyl oleate, ethyl linolenate, ethyl linoleate, Kojic acid, ascorbyl glucoside, erythromycin, clindamycin, metronidazole, gentamicin, fusidic acid, econazole, ketoconazole, mupirocin, neomocin, stretomicin, hydrogen peroxide, benzoil peroxide, cetylpyridinium, benzalkonium, chlorhexidin and relative salts and esters, silver and relative salts, both organic and inorganic, hydroxyacids and β hydroxyacids, both mono and bi carboxyls, such as glycolic acid, lactic acid (in the dextro and levorotatory forms and in racemic mixtures) hydroxybutyric acid (in the dextro and levorotatory forms and in racemic mixtures), mandelic acid (in the dextro and levorotatory forms and in racemic mixtures), tartaric acid (in the dextro and levorotatory forms and in racemic mixtures), malic acid (in the dextro and levorotatory forms and in racemic mixtures), salicylic acid, 3-hydroxybenzoin acid, 4-hydroxybenzoin acid, cysteine, acetyl cysteine, glycine, used singularly or in association with one or more including the relative salts, esters and amides and the relative D-L-DL forms.

5. A composition according to claim 4, wherein said additional substances are contained in a percentage by weight from 0.01% to 50%, preferably from 0.5 to 15%.

6. Use of a composition containing triethyl citrate according to claim 1 as a pharmaceutical substance at least for the treatment of cutaneous pathologies both directly and indirectly affected by infections of a bacterial origin.

7. Use according to claim 6 of a composition containing triethyl citrate in combination with an antibiotic for the treatment of cutaneous pathologies both directly and indirectly affected by infections of a bacterial origin.

8. Use of a composition containing triethyl citrate according to claim 1 as a cosmetic substance at least for the treatment of cutaneous blemishes both directly and indirectly caused by a bacterial component.

9. Method for the pharmaceutical or cosmetic cure of the skin including the procedures to use a composition containing triethyl citrate as an active ingredient, either pure or in combination with synergists, to formulate said composition in a preparation for external use and to apply said preparation for a length of time and using a sufficient quantity on the skin for treatment of cutaneous pathologies directly or indirectly affected by infections of a bacterial origin such as, pyodermatitis, dermatitis, eczema and cutaneous blemishes caused by a bacterial component.

10. A composition according to claim 2, containing the active ingredient represented by triethyl citrate in association with at least one of the additional substances chosen between trans-retinal acid, retinol, retinaldehyde, tocopherol, ascorbic acid, p-aminobenzoic acid, rutin, β-Carotene, tiamin, riboflavin, pyridoxine, pyridoxale, niacin, nicotinic acid, nicotinamide, pantothenic acid, pantenol, glucosamine, aceylglucosamine, folic acid, lecithin, phosphplipids such as, for example phosphatidylcholine, phosphatidylethanolamine, phosphatidic acid, lyso-phosphatidylcholine, hydroquinone, oleic acid, linoleic acid, linolenic acid, ethyl oleate, ethyl linolenate, ethyl linoleate, Kojic acid, ascorbyl glucoside, erythromycin, clindamycin, metronidazole, gentamicin, fusidic acid, econazole, ketoconazole, mupirocin, neomocin, stretomicin, hydrogen peroxide, benzoil peroxide, cetylpyridinium, benzalkonium, chlorhexidin and relative salts and esters, silver and relative salts, both organic and inorganic, hydroxyacids and β hydroxyacids, both mono and bi carboxyls, such as glycolic acid, lactic acid (in the dextro and levorotatory forms and in racemic mixtures) hydroxybutyric acid (in the dextro and levorotatory forms and in racemic mixtures), mandelic acid (in the dextro and levorotatory forms and in racemic mixtures), tartaric acid (in the dextro and levorotatory forms and in racemic mixtures), malic acid (in the dextro and levorotatory forms and in racemic mixtures), salicylic acid, 3-hydroxybenzoin acid, 4-hydroxybenzoin acid, cysteine, acetyl cysteine, glycine, used singularly or in association with one or more including the relative salts, esters and amides and the relative D-L-DL forms.

11. A composition according to claim 3, containing the active ingredient represented by triethyl citrate in association with at least one of the additional substances chosen between trans-retinal acid, retinol, retinaldehyde, tocopherol, ascorbic acid, p-aminobenzoic acid, rutin, β-Carotene, tiamin, riboflavin, pyridoxine, pyridoxale, niacin, nicotinic acid, nicotinamide, pantothenic acid, pantenol, glucosamine, aceylglucosamine, folic acid, lecithin, phosphplipids such as, for example phosphatidylcholine, phosphatidylethanolamine, phosphatidic acid, lyso-phosphatidylcholine, hydroquinone, oleic acid, linoleic acid, linolenic acid, ethyl oleate, ethyl linolenate, ethyl linoleate, Kojic acid, ascorbyl glucoside, erythromycin, clindamycin, metronidazole, gentamicin, fusidic acid, econazole, ketoconazole, mupirocin, neomocin, stretomicin, hydrogen peroxide, benzoil peroxide, cetylpyridinium, benzalkonium, chlorhexidin and relative salts and esters, silver and relative salts, both organic and inorganic, hydroxyacids and β hydroxyacids, both mono and bi carboxyls, such as glycolic acid, lactic acid (in the dextro and levorotatory forms and in racemic mixtures) hydroxybutyric acid (in the dextro and levorotatory forms and in racemic mixtures), mandelic acid (in the dextro and levorotatory forms and in racemic mixtures), tartaric acid (in the dextro and levorotatory forms and in racemic mixtures), malic acid (in the dextro and levorotatory forms and in racemic mixtures), salicylic acid, 3-hydroxybenzoin acid, 4-hydroxybenzoin acid, cysteine, acetyl cysteine, glycine, used singularly or in association with one or more including the relative salts, esters and amides and the relative D-L-DL forms.

12. Use of a composition containing triethyl citrate according to claim 2 as a pharmaceutical substance at least for the treatment of cutaneous pathologies both directly and indirectly affected by infections of a bacterial origin.

13. Use of a composition containing triethyl citrate according to claim 3 as a pharmaceutical substance at least for the treatment of cutaneous pathologies both directly and indirectly affected by infections of a bacterial origin.

14. Use of a composition containing triethyl citrate according to claim 4 as a pharmaceutical substance at least for the treatment of cutaneous pathologies both directly and indirectly affected by infections of a bacterial origin.

15. Use of a composition containing triethyl citrate according to claim 5 as a pharmaceutical substance at least for the treatment of cutaneous pathologies both directly and indirectly affected by infections of a bacterial origin.

16. Use of a composition containing triethyl citrate according to claim 2 as a cosmetic substance at least for the treatment of cutaneous blemishes both directly and indirectly caused by a bacterial component.

17. Use of a composition containing triethyl citrate according to claim 3 as a cosmetic substance at least for the treatment of cutaneous blemishes both directly and indirectly caused by a bacterial component.

18. Use of a composition containing triethyl citrate according to claim 4 as a cosmetic substance at least for the treatment of cutaneous blemishes both directly and indirectly caused by a bacterial component.

19. Use of a composition containing triethyl citrate according to claim 5 as a cosmetic substance at least for the treatment of cutaneous blemishes both directly and indirectly caused by a bacterial component.
